(19) United States
(12) Patent Application Publication
(10) Pub. No.: US 2024/0402184 A1
(43) Pub. Date: Dec. 5, 2024

(54) DUAL ENZYME AMPLIFICATION BASED COLORIMETRIC SENSOR SYSTEM FOR ON-SITE DETECTION OF PATHOGEN

(71) Applicant: KOREA RESEARCH INSTITUTE OF BIOSCIENCE AND BIOTECHNOLOGY, Daejeon (KR)

(72) Inventors: Eun Kyung LIM, Daejeon (KR); Ji Sun Ki, Daejeon (KR); Jaewoo Lim, Daejeon (KR); Taejoon Kang, Daejeon (KR); Juyeon Jung, Daejeon (KR)

(73) Assignee: KOREA RESEARCH INSTITUTE OF BIOSCIENCE AND BIOTECHNOLOGY, Daejeon (KR)

(21) Appl. No.: 18/666,454

(22) Filed: May 16, 2024

(30) Foreign Application Priority Data
May 30, 2023 (KR) 10-2023-0069568

(51) Int. Cl. G01N 33/58; C12N 9/22; C12Q 1/44; G01N 33/52; G01N 33/543; G01N 33/569

(52) U.S. Cl. CPC G01N 33/581; C12N 9/22; C12Q 1/44; G01N 33/52; G01N 33/54326; G01N 33/569; C12N 2310/20

(57) ABSTRACT

The present disclosure relates to a dual enzyme amplification-based colorimetric sensor system for on-site detection of pathogens. The colorimetric sensor system according to the present disclosure may comprise a combination of the CRISPR/Cas12a system with an enzymatic reaction of urease, thereby facilitating on-site detection of pathogens without separate analytical equipment by analyzing the color change through dual enzyme amplification. In addition, it is possible to selectively change the target by changing the crRNA sequence depending on the target pathogen to be detected, which has the advantage of being applicable to various types of pathogens without limitation. Further, the present disclosure can be used as a point of care service (PoC) system capable of detecting the genes of pathogens directly down to the sub-ng level without separate analysis equipment by applying the detection color value derived using the colorimetric sensor system according to the present disclosure to a smartphone application (app).

### CROSS-REFERENCE TO RELATED APPLICATIONS

This application claims priority to KR Patent Application No. 10-2023-0069568 filed on May 30, 2023, the entire disclosure of which is incorporated herein by reference.

### INCORPORATION BY REFERENCE OF SEQUENCE LISTING

The content of the electronically submitted sequence listing, file name: Q297596 sequence listing as filed; size: 21,976 bytes; and date of creation: Apr. 4, 2024, filed herewith, is incorporated herein by reference in its entirety.

### TECHNICAL FIELD

The present disclosure relates to a dual enzyme amplification-based colorimetric sensor system for on-site detection of pathogens.

### BACKGROUND ART

Airborne microorganisms (pathogens), such as viruses, fungi, and bacteria, present a substantial threat to the health of both humans and animals, imposing significant societal burden. For instance, the World Health Organization (WHO) reported that the severe acute respiratory syndrome coronavirus 2 (COVID 2), had infected over 178 million individuals as of June 2021 and led to 3.8 million deaths. Meanwhile, bacteria are a common microbial community that humans frequently encounter, and contamination by bacteria is also a frequent cause of airborne diseases. According to the WHO report, an estimated 10 million people worldwide suffer from diseases resulting from bacterial infections annually, constituting 20% of the total annual global mortality. Accordingly, in order to reduce the transmission rate of pathogens and prevent pathogen-related diseases, early and rapid diagnosis of pathogens is being actively researched.

Several techniques are currently widely used for pathogen detection, including culture, immunoassay, and polymerase chain reaction (PCR)-based systems. However, culture-based methods have the disadvantage of being susceptible to sample contamination, being time and resource intensive, and relying on phenotypic and biochemical characterization. Immunoassay and PCR methods offer high sensitivity and specificity, but have the drawback of necessitating expensive reagents and analytical equipment. Electrochemical technology-based methods are highly accurate, but face challenges related to poor uniformity, primarily attributed to the use of non-commercial electrodes. In particular, it is practically not easy to quickly detect and immediately identify bacteria in the air, so there is an urgent need for rapid, simple, and inexpensive bacterial detection methods.

Meanwhile, optical biosensors are a technology with unique advantages such as portability, low cost, and ease of use. Among these biosensors, colorimetric methods are highly suitable for on-site detection because the results can be read directly with the unaided eye. When combined with a smartphone, these colorimetric analysis systems may be used as a point of care service (PoC) system for instant analysis without the need for additional analytical equipment. More recent colorimetric technologies use, for detection, changes in the shape and size of plasmonic nanoparticles, such as gold nanoparticles, or use color changes resulting from pH and metabolite production using enzymes. However, the plasmonic nanoparticles have the disadvantages of low synthesis uniformity, complicated synthesis process, poor signal reproducibility, easy aggregation under conditions of pH, salt, etc., and high sensitivity to buffer conditions.

Therefore, the present inventor has developed and provided a dual enzyme amplification-based colorimetric sensor system capable of easily, specifically and sensitively identifying pathogens with the unaided eye using enzyme-based colorimetric changes.

### DISCLOSURE

### Technical Problem

An object of the present disclosure is to provide a kit for detecting a pathogen comprising: a CRISPR/Cas12a complex comprising a CRISPR/Cas12a protein, and a guide RNA including a region that binds to the CRISPR/Cas12a protein and a guide sequence that hybridizes to a target DNA; nanoparticles immobilized with urease-conjugated single-stranded DNA (ssDNA); and a pH indicator.

Another object of the present disclosure is to provide a method of detecting a pathogen, comprising: reacting a sample with the kit for detecting a pathogen; and confirming, when single-stranded DNA (ss-DNA) of the nanoparticles immobilized with the urease-conjugated ssDNA is cleaved by activation of the CRISPR/Cas12a complex, a color change of a solution by the cleaved urease.

### Technical Solution

The present disclosure will be described in detail as follows. Meanwhile, each description and embodiment disclosed in the present disclosure may be applied to each of the other descriptions and embodiments. In other words, all combinations of various elements disclosed in the present disclosure fall within the scope of the present disclosure. In addition, it cannot be considered that the scope of the present disclosure is limited by specific descriptions described below.

Further, terms used herein are merely used for illustration purposes, which should not be construed as limiting the present disclosure. Singular expressions include plural expressions unless the context clearly indicates otherwise. In the present specification, terms such as “comprise” or “have” are intended to designate the presence of features, numbers, steps, operations, components, parts, or combinations thereof described in the specification and it should not be understood as precluding the possibility of the presence or addition of one or more other features, numbers, steps, operations, components, parts, or combinations thereof.

Further, unless defined otherwise, all terms used herein, including technical or scientific terms, have the same meaning as commonly understood by one of ordinary skill in the art to which the embodiments belong. Terms such as those defined in commonly used dictionaries should be interpreted as having a meaning consistent with the meaning in the context of the related art, and unless explicitly defined in the present application, it is not to be construed in an idealized or overly formal sense.

Hereinafter, the present disclosure will be described in more detail.

Air, soil and water bacterial infections pose a serious threat to public health due to high transmission rate and wide spread. As a result, early and rapid detection of pathogens is critical for controlling bacterial infectious diseases, and current detection methods typically rely on complex laboratory-based procedures and large measuring instruments.

Therefore, the present disclosure aims to provide a practical visual assay based on the unaided eye or smartphone for pathogen detection by applying dual enzyme amplification including a combination of the CRISPR/Cas12a system and the urease enzyme.

As described above, in one general aspect, the present disclosure provides a kit for detecting a pathogen comprising: a CRISPR/Cas12a complex comprising a CRISPR/Cas12a protein, and a guide RNA including a region that binds to the CRISPR/Cas12a protein and a guide sequence that hybridizes to a target DNA; nanoparticles immobilized with urease-conjugated ssDNA; and a pH indicator.

Specifically, in the system (kit) according to the present disclosure, as shown in FIG. 1, CRISPR/Cas12a is activated by recognizing a specific sequence of the double-stranded DNA (dsDNA) gene of the pathogen. The activated CRISPR/Cas12a cleaves the urease-conjugated single-stranded DNA (SSDNA) immobilized on the nanoparticle, and the urease released thereby increases the pH and induces a change in the color of the pH indicator, enabling visual identification of the pathogen. Further, the system (kit) according to the present disclosure is applied to a smartphone application as shown in the lower part of FIG. 1 (smartphone-based sensing), so that when the target pathogen is not present, the color of the pH indicator (phenol red) does not change (yellow), which is displayed as “Negative” on the smartphone application screen, and when the target pathogen is present, the color of the pH indicator (phenol red) changes (magenta), which is displayed as “Positive” on the smartphone application screen, thereby making it easy to check the presence (detection) of the pathogen.

Cas12a is a type of CRISPR-Cas protein, which refers to a CRISPR protein with the activity of randomly cleaving untargeted single-stranded DNA (SSDNA) when activated by the detection of target DNA, and is used interchangeably with “CRISPRCas12a” or “CRISPR/Cas12a”. Molecular diagnostic methods based on gene editing using the activity of Cas12a are being developed. In order for the CRISPR/Cas12a complex to recognize a target gene or nucleic acid, it requires a thymine-rich sequence, such as 5′-TTN-3′ or 5′-TTTN-3′, in the target nucleic acid, called a protospacer adjacent motif (PAM) sequence. The PAM sequence is a unique sequence determined by the Cas12a protein and is considered when determining the target sequence of the CRISPR/Cas12a complex. For example, a protospacer sequence and a target sequence complementary thereto may be determined within a sequence adjacent to the PAM sequence.

Cas12a protein may be derived from the genus Candidatus, Lachnospira, Butyrivibrio, Peregrinibacteria, Acidominococcus, Porphyromonas, Prevotella, Francisella, Candidatus Methanoplasma, or Eubacterium. As one example, it may be derived from microorganisms such as Parcubacteria bacterium (GWC2011_GWC2_44_17), Lachnospiraceae bacterium (MC2017), Butyrivibrio proteoclasiicus, Peregrinibacteria bacterium (GW2011 GWA 33_10), Acidaminococcus sp. (BV3L6), Porphyromonas macacae, Lachnospiraceae bacterium (ND2006), Porphyromonas crevioricanis, Prevotelladisiens, Moraxella bovoculi (237), Smiihella sp. (SC_KO8D17), Leptospira inadai, Lachnospiraceae bacterium (MA2020), Francisella novicida (U112), Candidatus Methanoplasma termitum, Candidatus Paceibacter, Eubacterium eligens, or the like, but is not limited thereto. As another example, the Cas12a protein may be derived from Parcubacteria bacterium (GWC2011_GWC2_44_17), Peregrinibacteria bacterium (GW2011 GWA 33_10), Acidaminococcus sp. (BV3L6), Porphyromonas macacae, Lachnospiraceae bacterium (ND2006), Porphyromonas crevioricanis, Prevotella disiens, Moraxella bovoculi (237), Leptospirainadai, Lachnospiraceae bacterium (MA2020), Francisella novicida (U112), Candidatus Methanoplasmatermitum, or Eubacterium eligens, but is not limited thereto.

Here, the pathogen may be, for example, a bacterial pathogen. For example, the pathogen may be a strain such as Bacillus cereus, Staphylococcus aureus, Micrococcus luteus, Salmonella enterica, Escherichia coli, Citrobacter rodentium, Bordetella bronchiseptica, Acinetobacter baumannii, Pseudomonas aeruginosa, Enterobacter cloacae, Klebsiella pneumoniae, Morganella morganii, Salmonella typhimurium, Shigella dysenteriae, Yersinia enterocolitica, Acinetobacter calcoaceticus, Francisella tularensis, Legionella pneumophila, Proteus vulgaris, Proteus mirabilis, Stenotrophomonas maltophilia, Pasteurella multocida, Haemophilus parasuis, Actinobacillus pleuropneumoniae, Streptococcus suis, Mycoplasma pneumoniae, Mycoplasma hominis, Mycoplasma genitalium, Mycoplasma fermentans, Mycoplasma amphoriforme, Mycoplasma penetrans, Campylobacter jejuni, Borrelia burgdorferi, Brucella abortus, Brucella canis, Listeria monocytogenes, Rickettsia ricketsii, Yersinia pestis, or the like, but is not limited thereto.

Further, the pathogen may be a viral pathogen. For example, the virus may be, but is not limited to, African horse disease virus; African swine fever virus; Akabane virus; Banja virus; Calicivirus (e.g., human enteroviruses such as norovirus and sapovirus), cercopithecine herpesvirus 1; chikungunya virus; Classic swine fever virus; coronaviruses (e.g., severe acute respiratory syndrome (SARS), Middle East respiratory syndrome (MERS), severe acute respiratory syndrome coronavirus 2 (SARS-CoV-2)); dengue viruses such as serotypes 1 (DENV1) and 3 (DENV3), and related viruses such as chikungunya virus (CHIKV); Dugbe virus; Ebola virus; Encephalitis viruses, such as eastern equine encephalitis virus, Japanese encephalitis virus, Murray Valley encephalitis and Venezuelan equine encephalitis virus; equine morbillivirus; flavirus, flexal virus; foot-and-mouth disease virus; Germiston virus; goat pox virus; Hantaan or other Hanta viruses; Hendra virus; human immunodeficiency virus (HIV); influenza viruses (e.g., H1N1, H5N1, avian influenza viruses); Lassa fever virus; louping ill virus; lymphocytic choriomeningitis virus; poliovirus; potato virus; chickenpox virus; South American hemorrhagic fever virus; Variola major virus (smallpox virus); vesicular stomatitis virus; West Nile virus; yellow fever virus; a human-pathogenic flavivirus, such as Zika virus.

The system according to the present disclosure is a method for effectively detecting a target pathogen by producing a “guide RNA (crRNA)” that hybridizes with the genomic DNA (gDNA) of the target pathogen, and if the pathogen is one whose gDNA sequence is known, it is possible to produce a corresponding crRNA, which has the advantage that the target pathogen can be easily identified visually without being limited to a specific pathogen. In other words, the system according to the present disclosure has the advantage of enabling multiplexing of pathogens according to appropriate crRNA design.

In an embodiment according to the present disclosure, the crRNA may be designed to recognize the genomic DNA of a pathogen, preferably comprising a 5′ terminal TTTV PAM sequence. For example, the crRNA capable of being utilized to detect Bacillus cereus may have a sequence of SEQ ID NO: 1 (5′-UAA UUU CUA CUA AGU GUA GAU CTC AGG ATA TTG CTG CAT GTA-3′), the crRNA capable of being utilized to detect Micrococcus luteus may have a sequence of SEQ ID NO: 4 (5′-UAA UUU CUA CUA AGU GUA GAU CAC AGG TGG TAC TCA AGC AAT-3′), and the crRNA capable of being utilized to detect Staphylococcus aureus may have a sequence of SEQ ID NO: 7 (5′-UAA UUU CUA CUA AGU GUA GAU CGC TTG ATC TCG TTG GTG AGC-3′).

According to an embodiment of the present disclosure, when using the system according to the present disclosure, it is possible to visually clearly identify the presence of Bacillus cereus, Staphylococcus aureus, and Micrococcus luteus by a color change. Here, it was confirmed that the pathogens could be visually identified in the same way even if they were present in the air.

In other words, the system (kit) according to the present disclosure induces a colorimetric change in the solution by the target pathogen. Specifically, the system (kit) according to the present disclosure is a dual enzyme colorimetric detection method using 1) CRISPR/Cas12a enzyme and 2) urease, which utilizes the reaction of urease released by CRISPR/Cas12a activated in the presence of a pathogen by cleaving the bond between urease and ssDNA. In other words, the system (kit) utilizes the pH change caused by the ammonia produced as the released urease rapidly breaks down urea, and the corresponding change in pH is able to be visually identified with a pH indicator.

Therefore, the pH indicator may be, for example, any one selected from the group consisting of phenol red, cresol red, neutral red, and m-cresol purple, but may be used without limitation as long as it is an indicator showing a color change with pH change (basification).

The nanoparticles are not limited in type, as long as they are capable of capturing the urease-conjugated ssDNA, but as an example, may be any one selected from the group consisting of magnetic nanoparticles, lipid nanoparticles, silica nanoparticles, and disulfide nanoparticles. More preferably, for easy removal of impurities, magnetic nanoparticles may be used. Examples of magnetic nanoparticles may include metal nanoparticles such as iron nanoparticles, nickel nanoparticles, cobalt nanoparticles, gold nanoparticles, silver nanoparticles, and platinum nanoparticles, etc.

In particular, by using hierarchical metal nanoparticles (nanostructures), it is possible to adsorb pathogens onto the nanoparticles, effectively capturing the pathogens from the air, water, droplets, aerosols, or surfaces of objects.

Here, the nanoparticle may capture (fix) the urease conjugated-ssDNA through the combination of an avidin analog and biotin. Examples of the avidin analog may include streptavidin, neutravidin, and captavidin.

Furthermore, the urease-conjugated ssDNA may be used without limitation in type as long as it is an oligomer capable of conjugating urease to ssDNA. Examples thereof may include maleimide, succinic anhydride, and N-hydroxysuccinimide ester.

Meanwhile, the system (kit) according to the present disclosure may further comprise a device capable of analyzing the color of the solution. For example, the system may further comprise an absorption spectrum (absorbance) analysis device, a device for converting the spectrum to RGB colors, a device for converting the spectrum to HSV color tone, or a device for converting RGB color values to HSV color tone values. Furthermore, the system (kit) according to the present disclosure may be applied to a smartphone application that analyzes values converted by the device above and displays whether a pathogen is detected, thus providing easier identification information. Furthermore, a smartphone application custom-designed according to the present disclosure may diagnose a positive or negative condition of a disease by converting a smartphone camera captured image (optical output) into a color space mode regardless of the external environment (brightness, camera specifications).

According to an embodiment of the present disclosure, it was confirmed that the colorimetric detection system according to the present disclosure was capable of detecting even the sub-ng level of pathogen bacterial gDNA. In particular, even in the air capture model system, samples containing bacteria generated a signal difference of approximately twice that of the control group.

However, the color change according to urease concentration makes it difficult to distinguish the reaction by the color of the substrate solution observed at 1 to 4 μg/ml compared to the control group, which may make hinder accurate pathogen detection by the unaided eye.

For this problem, the concentration of the sample in the solution may be accurately and quantitatively determined by measuring the color change in absorbance and using the Beer-Lambert law. However, measurement of absorbance based on spectroscopic measurement generally requires a high-performance spectrum analysis device such as a multi-reader, which is less portable and bulkier, making it difficult for consumers to use in their daily lives.

Accordingly, in an embodiment of the present disclosure, colorimetric detection using a smartphone-based application is applied with the goal of observing a subtle change in the absorbance spectrum, i.e., a precise difference between the control group and a urease concentration (1 μg/mL) of 0.010 at 420 nm and 0.023 at 560 nm wavelength.

Typically, smartphone cameras have an RGB color sensor that collects an array of three primary colors. The RGB color model typically represents 8 bits from 0 to 255, with each color array recorded as 0 to 100% intensity. In other words, because one particular slide plane in the three-dimensional coordinates of the RGB color cube contains multiple mixed colors, comparisons of colors using RGB values employ vector operations between three-dimensional coordinates.

Meanwhile, the color space of the HSV model is represented in a cylindrical geometry, where the polar axis corresponds to S (saturation), the vertical axis corresponds to V (value), and the angular coordinate corresponds to H (hue). The HSV model is more advantageous than RGB for color detection under various observation conditions since the HSV model requires only the H value for color identification. In particular, since HSV model is able to obtain consistent H values under conditions of color brightness differences, it is possible to yield more flexible results for external illumination brightness than RGB color model, which makes HSV hue-based models potentially more advantageous for applications in smartphones compared to RGB color-based models.

The above-described kit may further comprise a user guide describing the optimal reaction performance procedures (conditions). The instructions are printed materials that describe the use of the kit, for example, the application order of the sample and kit contents. The instructions include brochures in the form of pamphlets or leaflets, labels affixed to the kit, and a description on the surface of the package containing the kit. Instructions also include information published or made available via an electronic medium, such as the Internet.

Another object of the present disclosure is to provide a method of detecting a pathogen, comprising: reacting a sample with the kit for detecting a pathogen; and confirming, when single-stranded DNA (ss-DNA) of the nanoparticles immobilized with the urease-conjugated ssDNA is cleaved by activation of the CRISPR/Cas12a complex, a color change of a solution by the cleaved urease.

More specifically, the present disclosure provides a method of detecting a pathogen, comprising: reacting a sample with a kit for detecting a pathogen, the kit comprising a CRISPR/Cas12a complex including a CRISPR/Cas12a protein, and a guide RNA including a region that binds to the CRISPR/Cas12a protein and a guide sequence that hybridizes to a target DNA; nanoparticles immobilized with urease-conjugated single-stranded DNA (SSDNA); and a pH indicator; performing a reaction in which ss-DNA of the nanoparticles immobilized with the urease-conjugated ssDNA is cleaved by activation of the CRISPR/Cas12a complex; and confirming a color change of a solution by the cleaved urease.

As used herein, the terms “target sample” and “sample” refer to any sample comprising any DNA/RNA and/or target gene.

As used herein, the term “biological sample” means any sample containing any RNA and/or target RNA. The biological sample may be any tissue or body fluid obtained from a subject.

The biological samples include, but are not limited to, subject's sputum, blood, serum, plasma, blood cells (e.g., white blood cells), tissue, biopsy samples, smears, lavage samples, swab samples, cell-containing fluids, free nucleic acids, urine, fluid peritoneal and pleural fluid, cerebrospinal fluid, feces, leakage fluid, or cells from these sources. Biological samples may also include tissue sections taken for histological purposes, i.e., frozen or fixed sections or microdissected cellular or extracellular portions thereof. The biological sample may be obtained by a method that does not adversely affect the subject.

These samples may originate from subjects suspected of being infected with pathogens.

In the present disclosure, the step of confirming a color change of a solution may be performed via a smart device. Specifically, it may be a portable smartphone or tablet PC. In addition to these devices, it may be any device capable of sensing information about the surrounding environment, biology, or the like, as well as communicating or sharing such information with other devices or systems, interacting with each other, or making autonomous judgments. The smart device can analyze the colorimetric results of the kit taken by a camera or the like via an application to provide information on whether infection is caused by the pathogen. The application may also provide the results of the analysis to the user. The user may be provided with the analysis results in visual or audible form, for example, via a display or speaker.

Thus, advantageously, the method may further comprise analyzing a color change result of the solution via an application to provide information on whether infection is caused by the pathogen.

According to an embodiment of the present disclosure, the detection method according to the present disclosure produces a spectral change from 430 to 560 nm that is observable to the unaided eye. In other words, the step of confirming a color change of a solution according to the present disclosure may be to determine the presence or absence of a spectral change at 430 to 560 nm.

According to the present disclosure, the step of confirming a color change of a solution by the cleaved urease may be to automatically confirm positive and negative pathogen detection results through images captured using a camera.

It is also possible to automatically distinguish between positive and negative pathogen detection results by applying the corresponding spectral changes (changes in hue values) to a smartphone application, specifically, by applying the colorimetric results of the kit to images captured using a camera, preferably a camera included in the smart device, for accurate and convenient analysis of the detection results.

As such, the detection method according to the present disclosure does not require any special measuring device, enabling convenient, rapid, and immediate detection of pathogens, and thus can be widely applied to diagnose infectious bacteria (pathogens) in air, water, and soil, effectively protecting public safety.

### Advantageous Effects

The colorimetric sensor system according to the present disclosure may comprise a combination of the CRISPR/Cas12a system with an enzymatic reaction of urease, thereby facilitating on-site detection of pathogens without separate analytical equipment by analyzing the color change through dual enzyme amplification. In addition, it is possible to selectively change the target by changing the crRNA sequence depending on the target pathogen to be detected, which has the advantage of being applicable to various types of pathogens without limitation.

Further, the present disclosure can be used as a point of care service (PoC) system capable of detecting the genes of pathogens directly down to the sub-ng level without separate analysis equipment by applying the detection color value derived using the colorimetric sensor system according to the present disclosure to a smartphone application (app).

### DESCRIPTION OF DRAWINGS

The patent or application file contains at least one drawing executed in color. Copies of this patent or patent application publication with color drawings(s) will be provided by the Office upon request and payment of the necessary fee.

FIG. 1 is a schematic diagram of colorimetric bacterial detection based on dual enzyme amplification using CRISPR/Cas12a and urease.

FIG. 2 shows the results of evaluating the DNase activity of Cas12a guided by crRNA (wherein (a) indicates a schematic diagram of fluorescence-based validation of CRISPR/Cas12a assay, (b) and (c) indicate fluorescence kinetics measurement results using dsDNA oligomers (b) and gDNA extracted from cultured bacteria (c), and (d) indicates fluorescence kinetics measurement results for bacterial gDNA detection extracted from airborne aerosols; Bacillus cereus: B. C., Staphylococcus aureus: S. A., Micrococcus luterus: M. L.).

FIG. 3 shows the colorimetric detection results of pathogens depending on urease concentration (wherein (a) indicates results of color change according to enzyme reaction for 5 minutes, (b) indicates results of color change according to enzyme reaction for 30 minutes, (c) indicates absorbance values according to enzyme reaction for 5 minutes, (d) indicates absorbance values according to enzyme reaction for 30 minutes, (e) indicates absorbance values at 430 nm and 560 nm according to enzyme reaction for 5 minutes, and (f) indicates absorbance values at 430 nm and 560 nm according to enzyme reaction for 30 minutes).

FIG. 4 is a graph showing the absorbance linear RGB color converted from the pathogen detection absorbance spectrum depending on urease concentration (wherein (a) indicates absorbance spectrum of urease concentration reacted with enzyme for 5 minutes, (b) indicates converted absorbance linear RGB color from absorbance spectrum, (c) indicates converted transmittance linear RGB color, and (d) indicates a graph showing converted absorbance linear RGB color from absorbance spectrum).

FIG. 5 shows the outcomes of comparing RGB and HSV color spaces converted from pathogen detection absorbance spectra depending on urease concentration (wherein (a) indicates outcomes of 3D RGB color cubes and the arrangement of mixed colors with specific G values at 0 μg/ml and 32 μg/ml urease, (b) indicates outcomes of cylindrical color space and the arrangement of mixed colors with specific H values at 0 μg/ml and 32 μg/ml urease, and (c) indicates HSV color graph converted from absorbance spectrum).

FIG. 6 shows the colorimetric detection results under various light conditions using urease (black background: conditions without light, and under light: conditions with high intensity of incandescent light).

FIG. 7 shows the results of smartphone-based pathogen colorimetric detection depending on urease concentration (wherein (a) indicates schematic diagram of smartphone application design for color recognition, (b) indicates a graph of hue values according to enzyme reaction for 5 minutes, and (c) indicates a graph of hue values according to enzyme reaction for 30 minutes).

FIG. 8 shows the results of smartphone-based colorimetric sensing (wherein (a) indicates an image of a substrate solution in the presence and absence of bacterial dsDNA oligomers, (b) indicates an absorbance graph, (c) indicates a graph of hue values of the substrate solution, and (d) indicates HSV color results obtained using a custom-designed application).

FIG. 9 shows the measurement results of HSV color values of the solution using a customized smartphone application when gDNAs of pathogens (Bacillus cereus, Staphylococcus aureus, and Micrococcus luteus) are expressed, respectively.

FIG. 10 shows the results of evaluating the detectability of actual bacteria using an airborne (aerosol) bacteria capture model (wherein (a) indicates measurement of sensitivity of dual enzyme colorimetric assay using CISPR/Cas12a and urease, (b) indicates confirmation of applicability to bacterial gDNA, and (c) indicates absorbance graphs (left) and corresponding image (right) of visible detection sensitivity using extracted bacterial gDNA, (wherein (i) indicates colorimetric detection images, (ii) indicates absorbance images of colorimetric analysis for detecting Bacillus cereus, (iii) indicates absorbance graphs of colorimetric analysis for detecting Staphylococcus aureus, and (iv) indicates absorbance graphs of colorimetric analysis for detecting Micrococcus luteus, respectively).

FIG. 11 shows the results of evaluating the detectability of Staphylococcus aureus (S. aureus) when introducing hierarchical gold nanostructures (wherein (a) indicates scanning electron microscope images of hierarchical gold nanostructures, (b) indicates scanning electron microscope images of hierarchical gold nanostructures containing Staphylococcus aureus adsorbed thereon, and (c) indicates absorbance values at 430 nm and 560 nm in the presence and absence of aerosol bacteria and a colorimetric detection image).

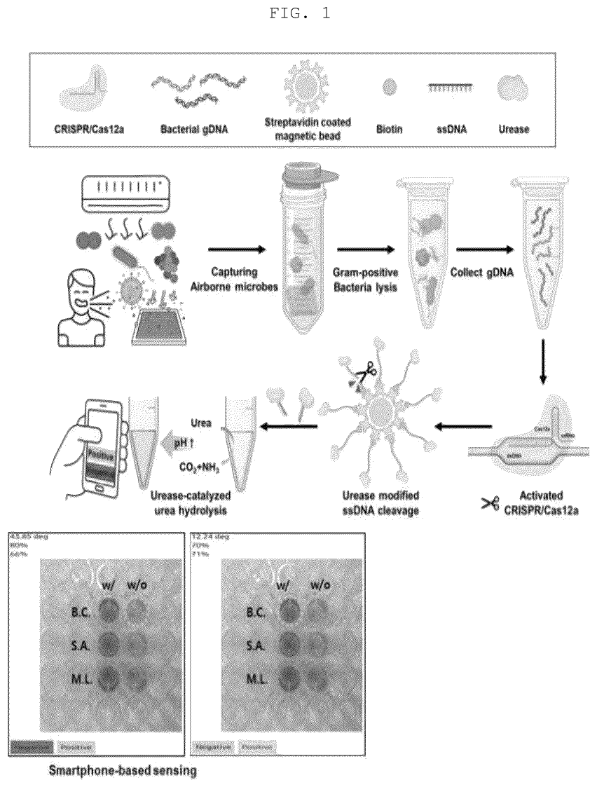

### BEST MODE

The following Examples are presented to enhance comprehension of the present disclosure. These Examples are only provided to more easily understand the present disclosure, and do not impose limitations on the content of the present disclosure.

### Experimental Materials and Methods

A. Preparation of Urease-Modified Single-Stranded DNA (urDNA)

Urease-modified single-stranded DNA (urDNA) was synthesized by combining urease with single-stranded oligodeoxynucleotide (ssDNA). First, a solution (100 μM) was prepared by dissolving ssDNA with amine and biotin modifications at the 5′- and 3′-termini in diethylpyrocarbonate (DEPC) and Sulfo-SMCC aqueous solution (6.4 mM). 10 μl of ssDNA (1 nM) and 7 μl of sulfo-SMCC (45 nM) were mixed, adjusted with PBS buffer to make the final volume 100 μl, and reacted at 25° C. for 1 hour.

Subsequently, excess sulfo-SMCC was removed by passing through a centrifugal filter (Merck Millipore, Villerica, MA, USA) with a molecular weight cut-off (MWCO) of 3 kDa to obtain maleimide-activated DNAs (maleimide-DNAs). Then, 10 μM of maleimide DNA resuspended in 100 μL of PBS and urease (1.5 mg) dispersed in 400 μL of PBS were mixed in pH 6.5 acidic buffer at 25° C. overnight to conjugate the maleimide DNAs and the thiol of urease. Thereafter, the mixture was filtered using a 100 kDa cut-off centrifugal column (Merck Millipore, Billerica, MA, USA) and then resuspended in 100 μl of PBS to obtain urease-modified single-stranded DNA (urDNA) at a concentration of 10 μM.

B. Fabrication of Magnetic Beads Immobilized with urDNA (MB@ urDNA)

300 μg of magnetic beads were washed twice with 500 μl of binding and washing buffer (B/W buffer; 5 mM Tris-HCl, 0.5 mM EDTA, 1 M NaCl, 0.05% Tween 20; pH 7.5). Then, 100 μM of urDNA was resuspended in 500 μl of B/W buffer and incubated for 2 hours with gentle rotation at 1 rpm. Afterward, only the urDNA-immobilized magnetic beads (MB@urDNA) were magnetically separated and dispersed in 500 μl of B/W buffer to obtain a final concentration of 10 mg/ml of MB@urDNA.

### C. Measurement of Nuclease Sensitivity (Collateral DNase Activity) Using Cas12a

The CRISPR/Cas12a solution was prepared by mixing equal volumes of 2 μM Lachnospiraceae bacterium Cas12a and 1 UM crRNA at 37° C. for 1 hour. In addition, 50 μl solutions containing 10 μl of various concentrations of dsDNA were prepared to determine the nuclease sensitivity (collateral DNase activity) of Cas12a based on fluorescence. Afterwards, 2 μl of reporter probe F-ssDNA-Q (50 UM) was mixed with 10 μl of cas12a/crRNA (0.5 μM), 5 μl of 10× NEbuffer 2.1, and 23 μl of DEPC-treated water. The fluorescence intensity of the solution was measured using a hybrid-multimode microplate reader and monitored at an excitation wavelength of 480 nm and an emission wavelength of 520 nm.

In addition, for colorimetric analysis using MB@urDNA, a reaction solution containing 50 μl solution (containing 5 μl of dsDNA of various concentrations), 30 μl of MB@urDNA (10 mg/ml), 10 μl of cas12a/crRNA (0.5 μM), and 5 μl of 10× NEbuffer 2.1 was prepared and incubated at 37° C. for 30 minutes. Afterwards, impurities were separated using a magnet, and 40 μl of the supernatant was transferred to a 96-well microplate. Then, 130 μl of substrate solution, 10 μl of PBS, 130 μl of urea substrate solution (2M NaCl, 60 mM MgCl₂, 50 mM urea, and 1 mM HCl) and 0.04% phenol red were added.

### D. Preparation of Pathogen Cultures

All pathogens (Bacillus cereus, Staphylococcus aureus, and Micrococcus luteus) used in the present experiment were provided by Korean Collection for Type Cultures (KCTC). Luria-Bertani solution and TSB solution were prepared by adding 5 g of LB and 6 g of TSB, respectively, with 200 ml of distilled water (DW) to autoclaved sterilized bottles. Luria-Bertani agar plates were prepared by adding 12.5 g of LB, 7.5 g of agar and 500 mL of distilled water.

Culture of pathogenic bacteria was prepared by mixing 15 ml LB broth and bacteria in a 50 ml conical tube and culturing overnight at 180 rpm in a shaking incubator at 37° C. Bacterial genomic DNA was extracted using the G-spin Genomic DNA Extraction kit (Cat. No. 17121) according to the manufacturer's instructions.

### E. Development of Smartphone Application for Pathogen Detection Using Colorimetric Analysis

The pathogen activation diagnostic application (app) was developed using MIT App Inventor, a visual programming environment that allows functional app programming for smartphones and tablets. The application according to the present disclosure acts as a means of pathogen detection/diagnosis based on color change (formation) as urease changes the absorbance of the medium.

### Example 1. Evaluation of DNase Activity of Cas12a Guided by crRNA

In order to prove the pathogen detection ability of the crRNA designed in the present disclosure, three types of gram-positive bacteria including Bacillus cereus, Staphylococcus aureus, and Micrococcus luteus were selected as pathogens.

First, to apply the CRISPR/Cas12a detection system according to the present disclosure, crRNA (CRISPR DNA) capable of recognizing pathogen genomic DNA containing the 5′ terminal TTTV PAM (protospacer adjacent motif) sequence is required. For the present experiment, crRNA showing low G-quadruplex formation and low overlap efficiency was selected using the CRISPRscan program (http://CRISPRscan.org).

Further, modified DNA oligonucleotides were purchased from Integrated DNA Technologies (IDT, Coralville, Lowa, USA) and Bioneer (Daejeon). Probe sequence information used in this experiment is shown in Table 1 below.

TABLE 1

| Bacteria | | 5′-sequence-3′ |
|---|---|---|
| Bacillus | crRNA | UAA UUU CUA CUA AGU GUA GAU CTC AGG ATA TTG CTG |
| cereus | (SEQ ID NO: 1) | CAT GTA |
| | TS | GGT ATC ATT TGC TCA GGA TAT TGC TGC ATG TAT GGT |
| | (SEQ ID NO: 2) | TGA T |
| | NTS | ATC AAC CAT ACA TGC |
| | (SEQ ID NO: 3) | TAC C |
| Micrococcus | crRNA | UAA UUU CUA CUA AGU GUA GAU CGC TTG ATC TCG TTG |
| luteus | (SEQ ID NO: 4) | GTG AGC |
| | TS | CGC GCT GTT TCC GCT TGA TCT CGT TGG TGA GCT CGG |
| | (SEQ ID NO: 5) | TGA C |
| | NTS | GTC ACC GAG CTC ACC AAC GAG ATC AAG CGG AAA CAG |
| | (SEQ ID NO: 6) | CGC G |
| Staphylococcus | crRNA | UAA UUU CUA CUA AGU GUA GAU CAC AGG TGG TAC TCA |
| aureus | (SEQ ID NO: 7) | AGC AAT |
| | TS | CAT CAT GAT TTA CAC AGG TGG TAC TCA AGC AAT TCA |
| | (SEQ ID NO: 8) | AAA T |
| | NTS | ATT TTG AAT TGC TTG AGT ACC ACC TGT GTA AAT CAT |
| | (SEQ ID NO: 9) | GAT G |

* Bases in bold indicate the PAM region/TS (target strand)/NTS (non target strand).

Specifically, with respect to the action mode of CRISPR/Cas12a according to the present experiments, complementary hybridization between genomic DNA (gDNA) of pathogenic bacteria (Bacillus cereus, Staphylococcus aureus, and Micrococcus luteus) and crRNA activates CRISPR/Cas12a. Here, the activated Cas12a acts as a nuclease in the vicinity of single-stranded DNA (ssDNA), cleaving the bond between the ssDNA and the fluorescent probe, enabling fluorescence detection.

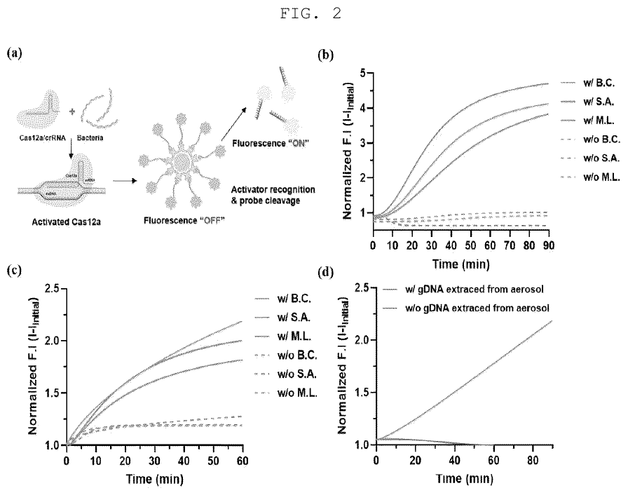

Based on this action mode of CRISPR/Cas12a, FAM-modified ssDNA was first immobilized on magnetic beads to evaluate the activity (activity of Cas12a) of DNase CRISPR/Cas12a in the presence of bacterial gDNA for crRNA sequence compatibility. As shown in (a) of FIG. 2, in the presence of pathogen gDNA, activated Cas12a cleaves ssDNA to generate a fluorescent signal.

The experimental results showed that the fluorescence intensity increased approximately 4.0-, 4.5-, and 3.8-fold in the presence of Bacillus cereus, Staphylococcus aureus, and Micrococcus luteus, respectively, as shown in (b) of FIG. 2. While the absolute intensity of the fluorescence signal varied slightly depending on the type of bacteria, the pattern and rate of increase were similar across all three conditions. These results also showed that even at 60 minutes of reaction time, the presence of Bacillus cereus, Staphylococcus aureus, and Micrococcus luteus increased fluorescence intensity by 3.7-, 4.3-, and 3.2-fold, respectively, compared to the control group.

Furthermore, since the results in (b) of FIG. 2 demonstrated that the reaction time of 60 minutes was sufficient, the reaction of CRISPR/cas12a with gDNA was observed for 60 minutes, and as shown in (c) of FIG. 2, the presence of gDNA from Bacillus cereus, Staphylococcus aureus, and Micrococcus luteus, increased the fluorescence intensity by 2.2-fold, 2.0-fold, and 1.8-fold, respectively, compared to the condition in which the corresponding gDNA was not present.

Finally, as shown in (d) of FIG. 2, the fluorescence kinetics were checked using a genetic sample of bacteria collected from the air, indicating that the fluorescence intensity increased by 1.7- and 2.25-fold compared to the control group when reacted for 60 and 90 minutes, respectively. These results demonstrate that the crRNAs in Table 1 designed in the present disclosure recognize the sequences of actual bacteria and activate CRISPR/Cas12a.

### Example 2. Construction of Smartphone-Based Colorimetric Analysis Method

When urease is present in a sample containing urea and phenol red, the urease hydrolyzes the urea and increases the pH, changing the color of the solution from yellow to magenta. Accordingly, a colorimetric detection method using urease was constructed to directly confirm the visual detection of pathogens and accurately determine the detection level.

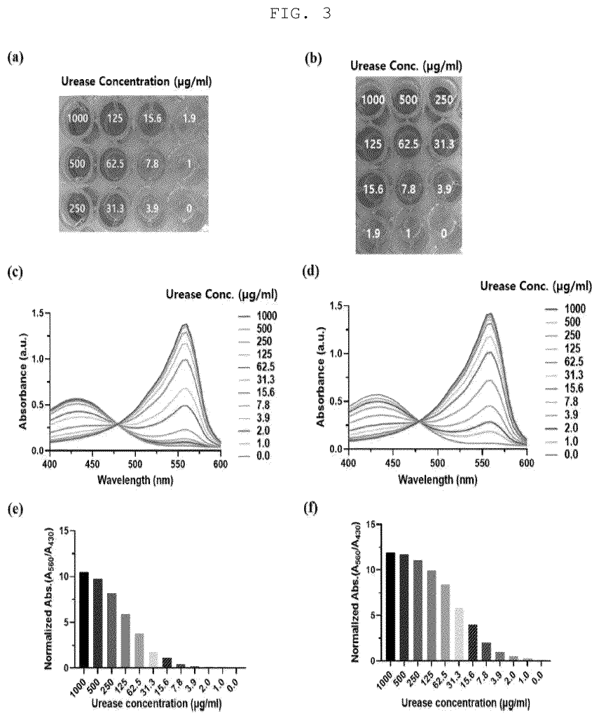

As a result of the experiment, the color difference was visually perceptible at urease concentrations of 3.9 μg/mL or more in a 5-minute reaction period as shown in (a) of FIG. 3, and the color of the solution at urease concentrations lower than 3.9 μg/mL was visually distinct in a 30-minute reaction period as shown in (b) of FIG. 3.

Meanwhile, the changes in visible color were related to differences in the absorption spectra, and each condition with different urease concentrations was distinguished by absorbance spectra (spectral analysis). In other words, as shown in (c) of FIG. 3 and (d) of FIG. 3, it was found that with the increase in the concentration of urease, the peak at 430 μm decreased and the peak at 560 μm increased. These results are due to the change in absorbance of phenol-red indicator in the solution when pathogens (bacteria) are present in the solution since the hydrolysis of urea by urease produces ammonia.

However, the absorption spectrum analysis as described above has the disadvantage that the absorbance intensity changes easily with a slight decrease in the concentration of phenol red or a slight change in the volume of the total solution, so as one strategy to reduce this experimental error, the absorbance ratios of 430 μm and 560 μm were analyzed as shown in (e) of FIG. 3 and (f) of FIG. 3, and the results were consistent with the above spectrum analysis.

Meanwhile, the absorption-based assay as described above has the advantage of being able to distinguish even low concentrations of urea, but its drawback lies in its portability, making immediate on-site detection challenging. Therefore, a smartphone-based colorimetric analysis method was developed to convert the spectrum into color for application to a smartphone application so that the detection can be easily identified using a smartphone application.

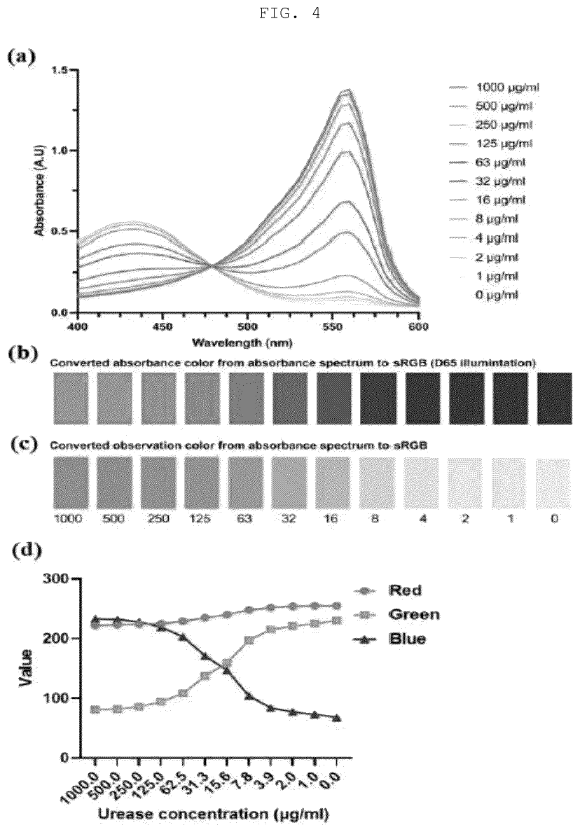

Since smartphone cameras typically acquire color images in the primary color array of red, green, and blue, as shown in FIG. 4, the spectral data was converted to RGB color values using the Commission Internationale de l′Elcairage (CIE) 1931 color model.

Specifically, (b) of FIG. 4 shows the result of converting the absorbance spectrum to linear RGB, which appears green and blue depending on the intensity of the prime wavelength (560 nm and 430 nm) regions, respectively. Observation color refers to reflected light, transmitted light, and scattered light, excluding the wavelength absorbed from incident light. Therefore, the observed color is the same as the inverted absorbance color as shown in (c) of FIG. 4. In other words, the observed color is consistent with values within the color range of the phenol red indicator from pH 6.0 to 8.0. Accordingly, it was observed that the color of the substrate solution changed depending on the urease concentration, which was influenced by the change in the primary wavelength of absorbance, as shown in (a) of FIG. 4. In addition, as shown in (d) of FIG. 4, the 430 nm and 560 nm absorbance spectra of the substrate solution changed with the concentration of urease, and thus the color change of the medium was more affected by jade and blue than red.

However, the RGB color model typically represents 8 bits from 0 to 255, with each color array recorded as 0 to 100% intensity. However, since the RGB color model is difficult to identify colors because the differences between colors are non-linear, comparative studies using specific primary colors among the RGB values may result in inaccurate color detection, and it is difficult to accurately observe the color change caused by urease concentration with the unaided eye. This is because it is difficult to distinguish the reaction by the color of the substrate solution observed at 1 to 4 μg/mL relative to the control group.

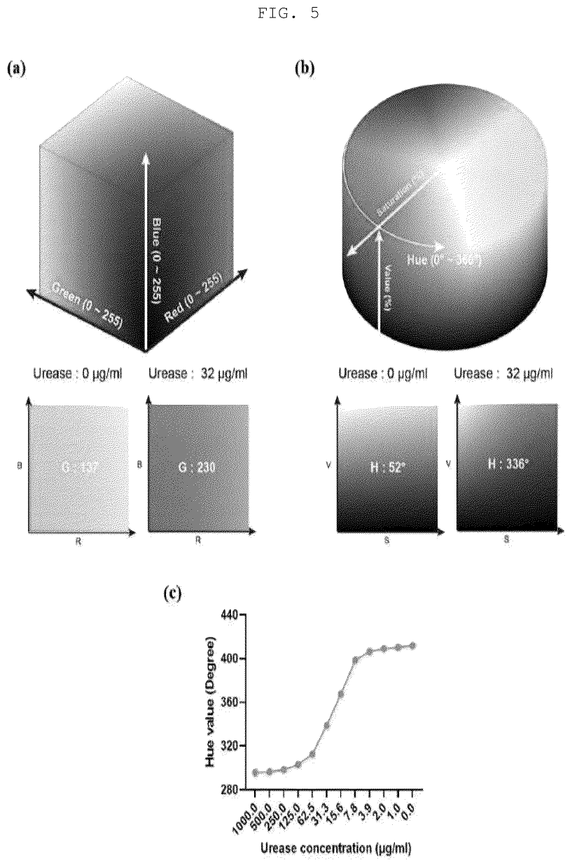

In other words, as shown in (a) FIG. 5 and (b) of FIG. 5, because one particular slide plane in the three-dimensional coordinates of the RGB color cube contains multiple mixed colors, comparisons of colors using RGB values employ vector operations between three-dimensional coordinates. In contrast, the color space of the HSV model is represented in a cylindrical geometry, where the polar axis corresponds to S (saturation), the vertical axis corresponds to V (value), and the angular coordinate corresponds to H (hue). Since the HSV model requires only the H value for color identification, it is more advantageous than RGB for color detection under various observation conditions. In particular, since HSV model is able to obtain consistent H values under conditions of color brightness differences, it is possible to yield more flexible results for external illumination brightness than RGB color model, and thus attempts were made to use the HSV color model.

As shown in (c) of FIG. 5, the HSV color models converted from the absorption spectrum were found to be capable of detecting color accurately enough to distinguish different concentrations of urease.

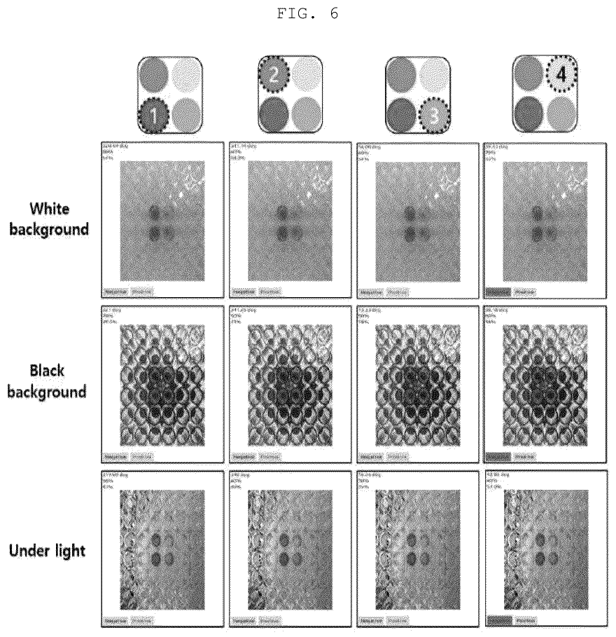

In addition, as shown in FIG. 6 and Table 2 below, the HSV color models converted from the absorption spectrum may allow for intuitive interpretation of color compared to the RGB color models because the color values do not change depending on the brightness and darkness of the measurement backgrounds or the presence or absence of light, thereby being applicable to various environments.

TABLE 2

| | 1 | 2 | 3 | 4 |
|---|---|---|---|---|
| White background | 320.49° | 341.14° | 16° | 39.43° |
| Black background | 321° | 341.25° | 13.33° | 38.18° |
| Under light | 321.82° | 342.86° | 18.75° | 37.50° |

Therefore, the color was analyzed by converting RGB to HSV values through the CIE color model expressed in Equation 1 below, and results thereof are shown in Table 3 below. In other words, Table 3 shows the RGB and HSV values converted from the absorption spectra after reacting with various concentrations of urease for 5 minutes.

$$[ Calculation 1 ]$$

$$H = \{ \begin{matrix}0 ° , & Δ = 0 & & \\ 60 ° \times ( \frac{{G}^{′} - {B}^{′}}{Δ} mod 6 ) , & {C}_{max} = {R}^{′} & & S = \{ \begin{matrix}0 , & {C}_{max} = 0 \\ \frac{Δ}{{C}_{max}} , & {C}_{max} \neq 0\end{matrix} \\ 60 ° \times ( \frac{{B}^{′} - {R}^{′}}{Δ} + 2 ) , & {C}_{max} = {{G}^{′}}^{↵} & & \\ 60 ° \times ( \frac{{R}^{′} - {G}^{′}}{Δ} + 4 ) , & {C}_{max} = {B}^{′} & & V = {C}_{max}\end{matrix}$$

* wherein [R′, G′, B′] represents the normalized values ranging from 0 to 1 for [R, G, B], Cmax are the maximum and minimum values of [R′, G′, B′], and Δ is Cₘₐₓ−Cₘᵢₙ. H values are in degrees, while S and V values are typically expressed in %.

TABLE 3

| | Conc. (μg/ml) | | | | | | | | | | | |
|---|---|---|---|---|---|---|---|---|---|---|---|---|
| | 1000 | 500 | 250 | 125 | 63 | 32 | 16 | 8 | 4 | 2 | 1 | 0 |
| R | 222 | 223 | 224 | 225 | 229 | 235 | 240 | 248 | 252 | 254 | 255 | 255 |
| G | 81 | 82 | 86 | 94 | 108 | 137 | 159 | 197 | 215 | 221 | 225 | 230 |
| B | 233 | 232 | 228 | 219 | 203 | 171 | 147 | 104 | 84 | 77 | 73 | 68 |
| H | 295.67 | 296.30 | 298.30 | 303.12 | 312.73 | 338.90 | 367.68 | 398.61 | 406.51 | 408.96 | 410.18 | 411.91 |
| S | 65.5% | 64.7% | 62.5% | 58.4% | 52.8% | 41.7% | 38.8% | 58.0% | 66.6% | 69.8% | 71.5% | 73.5% |
| V | 91.5% | 90.9% | 89.3% | 88.3% | 89.6% | 92.0% | 94.0% | 97.3% | 99.0% | 99.6% | 100.0% | 100.0% |

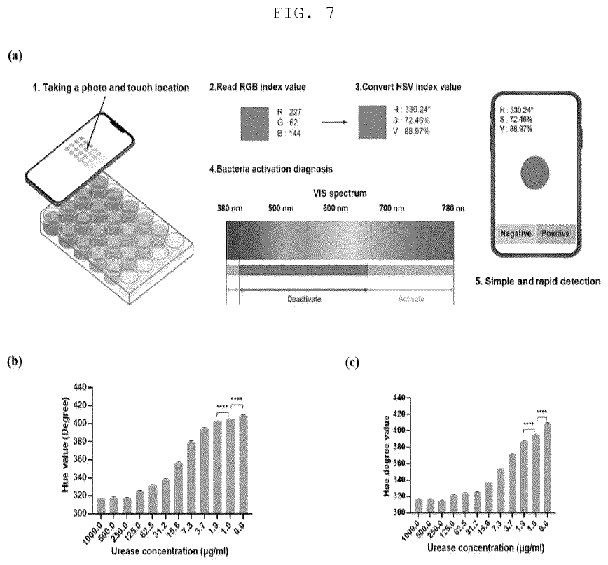

From the color value data shown in Table 3 above, color values from 300° to) 395° (35° were set to positive numbers, and an application was designed based on this setting. A functional schematic of the application according to the present disclosure is shown in (a) of FIG. 7. As shown in (b) of FIG. 7 and (c) of FIG. 7, the results of measuring the solution using the customized smartphone application were consistent with the absorbance graph and HSV color values. Therefore, the HSV color models converted from the absorption spectra were determined to be a suitable color analysis method for smartphone.

### Example 3. Smartphone-Based Colorimetric Sensing

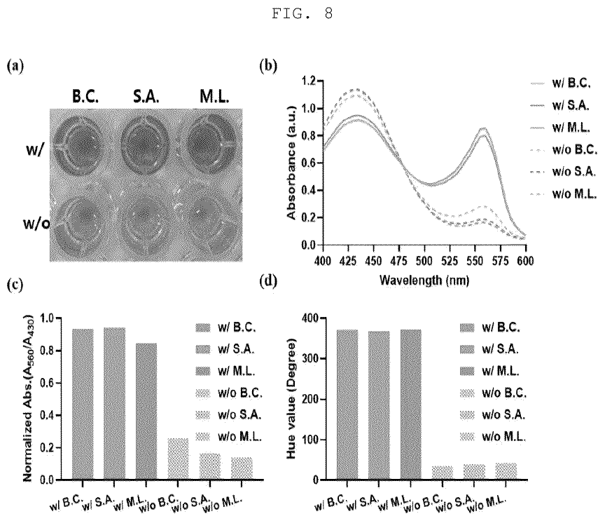

In the present experiment, a method for detecting pathogens using magnetic beads immobilized with urease-modified ssDNA was investigated. As shown in (a) of FIG. 8, activated CRISPR/Cas12a cleaved urease-modified ssDNA in the presence of genes from pathogenic bacteria (Bacillus cereus, Staphylococcus aureus, and Micrococcus luteus), and the released urease changed the color of the solution. This color change is converted to a hue value via a smartphone application, as described above, and this data may be used to determine the presence of pathogens.

In other words, as shown in (b) of FIG. 8, the absorbance graph shows that in the presence of the bacterial targeting oligomers, the intensity at 560 μm was 3.00, 4.57, and 4.88 times higher than the control group for Bacillus cereus, Staphylococcus aureus, and Micrococcus luteus, respectively, indicating the presence of pathogens with high sensitivity.

In addition, as shown in (c) of FIG. 8, the ratios of 560 nm to 460 nm were consistent even in the presence of bacterial gDNA. As shown in (d) of FIG. 8, the hue values of Bacillus cereus, Staphylococcus aureus, and Micrococcus luteus through the smartphone application were 11.43°, 8.57°, and 12.34°, respectively, and the unwrapped values were analyzed as 371.43°, 368.57°, and 372.34°.

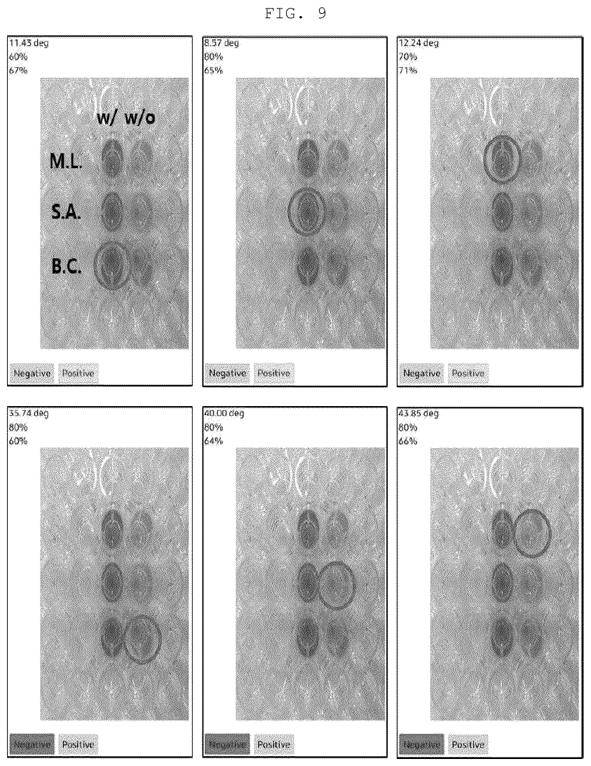

These results are divided into positive and negative based on the measured hue values on the smartphone screen, as shown in FIG. 9, where a positive result is displayed when the pathogen is present on the phone screen image and a negative result is displayed when the pathogen is not present thereon.

### Example 4. Evaluation of Actual Pathogen Detectability with Aerosol Pathogen Capture Model

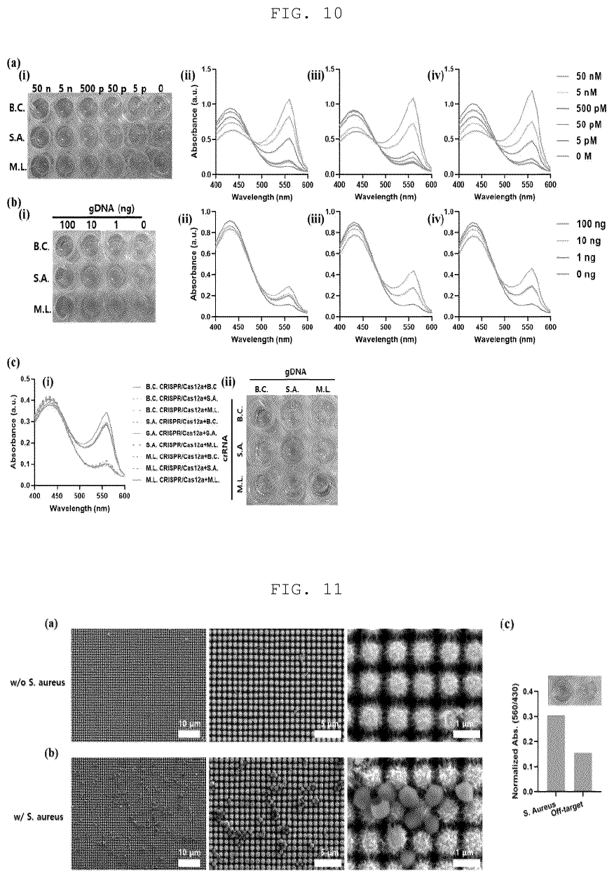

In the present experiment, the dual enzyme colorimetric assay was evaluated using an airborne (aerosol) pathogen capture model. As shown in (a) of FIG. 10, the reaction signal decreased as the concentration of synthetic bacterial dsDNA decreased from 50 nM to 0 M. The image of the reaction solution showed that the color changed from crimson to yellow as the concentration of the bacterial gene decreased ((a) (i) of FIG. 10). In the absorbance graph for each type of bacteria, depending on the concentration, the intensity at 560 nm decreased and the intensity at 430 nm increased ((a) (ii) to (a) (iv) of FIG. 10). In other words, the intensity at 560 nm absorbance was 1.58-, 1.42-, and 1.33-fold higher for Bacillus cereus, Staphylococcus aureus, and Micrococcus luteus, respectively, when the gene concentration was 5 μM compared to 0 M. These results demonstrate that the assay according to the invention detects genes of all types of pathogens at a concentration of 5 μM, even though the reactivity varies slightly depending on the type of pathogen.

Meanwhile, as shown in (b) of FIG. 10, the practicality of the colorimetric detection system according to the present disclosure was confirmed using genomic DNA extracted from actual bacteria rather than synthesized oligomers. Consequently, a color change could be observed with the unaided eye in solutions diluted 10-fold and 100-fold at 100 ng ((b) (i) of FIG. 10). The absorbance graphs for each type of bacteria also showed that the reactivity varied slightly different depending on the type of bacteria; however, all of them showed different colorations and absorbance spectra up to 1 ng, where the absorbance intensities at 560 nm for 1 ng of B. C., S. A., and M. L. were 1.66-, 2.25-, and 2.34-fold higher, respectively, than those for Ong ((b) (ii) to (b) (iv) of FIG. 10).

(c) of FIG. 10 shows the cross-reactivity of the three types of bacteria (Bacillus cereus, Staphylococcus aureus and Micrococcus luteus) used to evaluate the selectivity of the assay according to the present disclosure. Under the condition that the target gene and CRISPR/Cas12a are matched, the maximum absorbance intensities of B. C., S. A., and M. L. were 3.14, 2.99, and 3.37, respectively, and the assay according to the present disclosure did not respond to conditions that did not match each crRNA, and color and graph changes were only seen for conditions that did match.

Meanwhile, as shown in FIG. 11, hierarchical gold nanostructures were introduced to establish a model for capturing pathogens in air, soil, and water, and specifically, pathogens were adsorbed onto sea urchin-like alloy nanostructures, and the nanostructures were heated at 95° C. for 10 min to detect eluting bacterial genes. As Staphylococcus aureus causes purulent inflammation and food poisoning, has diverse routes of infection, and is classified as harmful bacteria, S. A. was used as an experimental model.

As shown in (b) of FIG. 11, it was confirmed through a scanning electron microscope that Staphylococcus aureus was adsorbed on the surface of the nanostructure. Further, the Staphylococcus aureus-adsorbed nanostructures were heated at 95° C. for 10 min to detect eluting bacterial genes, and as shown in (c) of FIG. 11, the absorbance ratio values of 560 and 430 nm for conditions with and without bacteria were 0.31 and 0.16, respectively. Therefore, it was confirmed that the dual enzyme amplification assay according to the present disclosure could effectively detect pathogens in aerosols using the bacterial adsorption model.

1. A kit for detecting a pathogen comprising:
a CRISPR/Cas12a complex comprising a CRISPR/Cas12a protein, and a guide RNA including a region that binds to the CRISPR/Cas12a protein and a guide sequence that hybridizes to a target DNA;
nanoparticles immobilized with urease-conjugated single-stranded DNA (ssDNA); and
a pH indicator.

2. The kit of claim 1, wherein the pH indicator is any one selected from the group consisting of phenol red, cresol red, neutral red, and m-cresol purple.

3. The kit of claim 1, wherein the pathogen is a bacterial pathogen or a viral pathogen.

4. The kit of claim 1, wherein the kit induces a colorimetric change in a solution by a target pathogen.

5. The kit of claim 1, wherein the nanoparticles are selected any one from the group consisting of magnetic nanoparticles, lipid nanoparticles, silica nanoparticles, and disulfide nanoparticles.

6. The kit of claim 5, wherein the magnetic nanoparticles are any one selected from the group consisting of iron nanoparticles, nickel nanoparticles, cobalt nanoparticles, gold nanoparticles, silver nanoparticles, and platinum nanoparticles.

7. The kit of claim 1, wherein the nanoparticles are immobilized with the urease-conjugated ssDNA via binding of an avidin analog and biotin.

8. The kit of claim 7, wherein the avidin analog is streptavidin, neutravidin, or captavidin.

9. The kit of claim 1, wherein the urease-conjugated ssDNA is formed by conjugating urease with the ssDNA via any one oligomer selected from the group consisting of maleimide, succinic anhydride, and N-hydroxysuccinimide ester.

10. The kit of claim 1, further comprising a device capable of analyzing a color of a solution.

11. The kit of claim 1, wherein the kit captures the pathogen from the air, water, droplets, aerosols, or surface of an object by adsorbing the pathogen onto a hierarchical metallic nanostructure.

12. A method of detecting a pathogen, comprising:
reacting a sample with a kit for detecting a pathogen, the kit comprising a CRISPR/Cas12a complex including a CRISPR/Cas12a protein, and a guide RNA including a region that binds to the CRISPR/Cas12a protein and a guide sequence that hybridizes to a target DNA; nanoparticles immobilized with urease-conjugated single-stranded DNA (ssDNA); and a pH indicator;
performing a reaction in which SS-DNA of the nanoparticles immobilized with the urease-conjugated ssDNA is cleaved by activation of the CRISPR/Cas12a complex; and
confirming a color change of a solution by the cleaved urease.

13. The method of claim 12, wherein the sample is any tissue or body fluid obtained from a subject.

14. The method of claim 12, wherein in the confirming of a color change of a solution by the cleaved urease, positive and negative pathogen detection results are automatically confirmed through images captured using a camera.

15. The method of claim 12, further comprising analyzing a color change result of the solution via an application to provide information on whether infection is caused by the pathogen.

16. The method of claim 12, wherein the pH indicator is any one selected from the group consisting of phenol red, cresol red, neutral red, and m-cresol purple.

17. The method of claim 12, wherein the nanoparticles are selected any one from the group consisting of magnetic nanoparticles, lipid nanoparticles, silica nanoparticles, and disulfide nanoparticles.

18. The method of claim 12, wherein the nanoparticles are immobilized with the urease-conjugated ssDNA via binding of an avidin analog and biotin.

19. The method of claim 12, wherein the urease-conjugated SSDNA is formed by conjugating urease with the ssDNA via any one oligomer selected from the group consisting of maleimide, succinic anhydride, and N-hydroxysuccinimide ester.

20. The method of claim 12, wherein the kit captures the pathogen from the air, water, droplets, aerosols, or surface of an object by adsorbing the pathogen onto a hierarchical metallic nanostructure.


SEQUENCE LISTING (9 sequences)

SEQ ID NO: 1 - DNA, 42 bp, synthetic construct
  misc_feature 1..22: note = RNA
  misc_feature 23: note = DNA
  misc_feature 24..28: note = RNA
  misc_feature 29: note = DNA
  misc_feature 30: note = RNA
  misc_feature 31..32: note = DNA
  misc_feature 33..34: note = RNA
  misc_feature 35: note = DNA
  misc_feature 36..38: note = RNA
  misc_feature 39: note = DNA
  misc_feature 40: note = RNA
  misc_feature 41: note = DNA
  misc_feature 42: note = RNA
  source 1..42: mol_type = other DNA
taatttctactaagtgtagatctcaggatattgctgcatgta

SEQ ID NO: 2 - DNA, 40 bp, Bacillus cereus
  source 1..40: mol_type = genomic DNA
  misc_feature 8..11: note = PAM region
ggtatcatttgctcaggatattgctgcatgtatggttgat

SEQ ID NO: 3 - DNA, 40 bp, Bacillus cereus
  source 1..40: mol_type = genomic DNA
atcaaccatacatgcagcaatatcctgagcaaatgatacc

SEQ ID NO: 4 - DNA, 42 bp, synthetic construct
  misc_feature 1..24: note = RNA
  misc_feature 25..26: note = DNA
  misc_feature 27..28: note = RNA
  misc_feature 29: note = DNA
  misc_feature 30: note = RNA
  misc_feature 31: note = DNA
  misc_feature 32..33: note = RNA
  misc_feature 34..35: note = DNA
  misc_feature 36..37: note = RNA
  misc_feature 38: note = DNA
  misc_feature 39..42: note = RNA
  source 1..42: mol_type = other DNA
taatttctactaagtgtagatcgcttgatctcgttggtgagc

SEQ ID NO: 5 - DNA, 40 bp, Micrococcus luteus
  source 1..40: mol_type = genomic DNA
  misc_feature 8..11: note = PAM region
cgcgctgtttccgcttgatctcgttggtgagctcggtgac

SEQ ID NO: 6 - DNA, 40 bp, Micrococcus luteus
  source 1..40: mol_type = genomic DNA
gtcaccgagctcaccaacgagatcaagcggaaacagcgcg

SEQ ID NO: 7 - DNA, 42 bp, synthetic construct
  misc_feature 1..27: note = RNA
  misc_feature 28: note = DNA
  misc_feature 29..30: note = RNA
  misc_feature 31: note = DNA
  misc_feature 32..33: note = RNA
  misc_feature 34: note = DNA
  misc_feature 35..41: note = RNA
  misc_feature 42: note = DNA
  source 1..42: mol_type = other DNA
taatttctactaagtgtagatcacaggtggtactcaagcaat

SEQ ID NO: 8 - DNA, 40 bp, Staphylococcus aureus
  source 1..40: mol_type = genomic DNA
  misc_feature 9..12: note = PAM region
catcatgatttacacaggtggtactcaagcaattcaaaat

SEQ ID NO: 9 - DNA, 40 bp, Staphylococcus aureus
  source 1..40: mol_type = genomic DNA
attttgaattgcttgagtaccacctgtgtaaatcatgatg